(19) United States
(12) Patent Application Publication
(10) Pub. No.: US 2026/0139231 A1
(43) Pub. Date: May 21, 2026

(54) CRYOPRESERVING AGENT OBTAINABLE FROM POLLEN OR SPORES

(71) Applicant: UNIVERSITY OF WARWICK, Coventry, Warwickshire (GB)

(72) Inventors: Thomas F. WHALE, Coventry, Warwickshire (GB); Kathryn A. MURRAY, Coventry, Warwickshire (GB); Nina L. H. KINNEY, Coventry, Warwickshire (GB); Matthew I. GIBSON, Coventry, Warwickshire (GB)

(21) Appl. No.: 18/706,305

(22) PCT Filed: Nov. 2, 2022
(86) PCT No.: PCT/GB2022/052763

§ 371 Date: Apr. 30, 2024

(30) Foreign Application Priority Data
Nov. 3, 2021 (GB) 2115794.6

(51) Int. Cl. C12N 5/00

(52) U.S. Cl. CPC C12N 5/562; C12N 5/525

(57) ABSTRACT

The present invention relates to cryopreservation of biological material. In particular, the invention provides processes for producing frozen cryopreserving compositions comprising biological material wherein the cryopreserving composition comprises ice-nucleating agents obtained or obtainable from pollen or spores. The invention also provides cryopreserving compositions comprising ice-nucleating agents obtained or obtainable from pollen or spores, together with a cryoprotectant; and cryopreserved compositions comprising biological material.

The present invention relates to cryopreservation of biological material. In particular, the invention provides processes for producing frozen cryopreserving compositions comprising biological material wherein the cryopreserving composition comprises ice-nucleating agents obtained or obtainable from pollen or spores. The invention also provides cryopreserving compositions comprising ice-nucleating agents obtained or obtainable from pollen or spores, together with a cryoprotectant; and cryopreserved compositions comprising biological material.

Cryopreservation is an essential process in clinical, industrial and research settings, enabling long term storage of biological material. Whilst cryopreservation is routine for many cell types, there are numerous instances where cryopreservation outcomes are inadequate for practical applications. For instance, cryopreserving cells of any type in the industry-standard 96-well plate format leads to significantly reduced post-thaw yields compared to vial based freezing, but is essential for high-throughput screening, as well as basic biomedical research.¹⁻³

The goal of any cryopreservation procedure is to cool biological material to a temperature low enough to inhibit chemical and biological processes.

There are, broadly speaking, two methods employed for cryopreservation: controlled rate (or ‘slow’ freezing)⁴ and vitrification, where very fast cooling rates are used to completely avoid ice formation.⁵

The present invention concerns controlled rate or slow freezing, which is the most commonly used approach for storage of cell lines. Controlled rate or slow freezing involves cooling at a known, steady rate, optionally in the presence of at least one cryoprotectant, which for nucleated cells is most commonly DMSO. The success of this approach relies on cooling fast enough to minimize the amount of time for which cells are exposed to potentially cytotoxic cryoprotectants and increased solute concentrations, while slow enough to allow cells to dehydrate, reducing the probability of fatal intracellular ice formation.^{6,7} Cell dehydration occurs because extra-cellular ice is more thermodynamically stable than supercooled intracellular liquid water at temperatures below the melting point of water in the system, meaning extracellular ice grows at the expense of the intracellular water. The balance of these two factors gives optimal outcomes at some intermediate cooling rate, typically around 1° C./minute for mammalian cells,⁸ although faster rates are also sometimes optimal, dependent on cell type and cryoprotectant type, among other factors.⁷

An often-neglected factor for successful controlled rate or slow freezing is the desirability of extracellular ice nucleation at relatively warm supercooled temperatures. The original studies on controlled rate freezing^{4,9} involved careful control of nucleation at temperatures near to the melting point of water by seeding with ice crystals, and manual control of nucleation temperature has since been shown to be beneficial,¹⁰ but has nevertheless often been overlooked. For small volumes such as 96-well plates, deliberate control is critical as smaller volumes of water have a much greater propensity to supercool.^{3,11} Ice nucleation at warm temperatures helps cells survive cryopreservation by increasing the opportunity for cell dehydration, minimizing the likelihood of damaging intracellular ice formation.¹⁰ If nucleation is left uncontrolled and occurs at cold temperatures, cells have less opportunity to dehydrate.

Numerous methods for inducing ice nucleation in cryopreservation procedures have been developed¹⁰; however, all these methods require direct intervention with the material being cryopreserved and are not suited to scalable freezing of biological samples. For instance, crystals of ice can be introduced into cryopreservation solutions at warm super-cooled temperatures, as was done in early studies on mammalian tissue culture.^{4,9} More recently, seeding has been used to improve cryopreservation outcomes in cell cultures frozen in the 96-well plate format.³ Electro-freezing, where strong electric fields induce ice formation via a poorly understood mechanism, has been used^{12,13} as has laser induced nucleation.¹⁴ A small number of chemical nucleants, which are capable of nucleating ice near the melting point via heterogeneous nucleation, have also been deployed. These include Snomax™,^{15,16} which is derived from plant pathogenic bacteria, cholesterol crystals,¹⁷ encapsulated silver iodide,¹³ biologically inert minerals¹⁹ and sand.²⁰ However, existing chemical nucleants are usually insoluble and generally difficult to sterilize, or else are difficult to introduce into cryopreservation volumes in a reproducible fashion¹⁰ which has led to encapsulation approaches and other workarounds. At present, there is no soluble, sterilisable nucleant suitable for cryopreservation which can be used without troublesome alteration to existing protocols.

The ability of windborne pollen grains to nucleate ice has been investigated by atmospheric scientists²¹⁻³⁰ because these pollens are present in the atmosphere in significant concentrations and may be able to influence cloud properties.

One hypothesized function of the pollen is that it serves to trigger precipitation in clouds, which may help the pollen contact other plants and fulfil its reproductive function.³⁰

The inventors have now demonstrated that aqueous extracts from certain plants (e.g. pollen washing water, PWW) increase the ice nucleation temperatures in 96-well plates from about −13° C. (water) to about −7° C. (PWW), indicating that PWW can be used in cryopreserving compositions. Application of PWW to the cryopreservation of immortalized T-lymphocyte cells in a 96-well plate format resulted in a significant increase in post-thaw metabolic activity from 63.9% to 97.4% when cooled at 2° C./min. When applied in a similar manner to the challenging process of cryopreserving A549 cell monolayers in 96-well plates, use of PWW increased post-thaw metabolic activity from 1.7% to 55.1%. Substantially improved outcomes were also found for both cell types using faster and slower cooling rates. Unlike other nucleators, PWW is soluble (which is a major benefit for automated or high throughput cryopreservation), has low cytotoxicity and is sterilisable. Such extracts can be regarded as a unique class of soluble cryoprotectant which act by inducing ice nucleation at warm temperatures.

Such extracts can easily be incorporated into existing methods with the aim of improving outcomes, particularly where nucleation temperature or variability is presently a limiting factor.

It is one object of the invention to provide a process for producing a frozen cryopreserving composition comprising biological material wherein the cryopreserving composition comprises ice-nucleating agents obtained or obtainable from pollen or spores. It is another object of the invention to provide a cryopreserving composition comprising ice-nucleating agents obtained or obtainable from pollen or spores, together with a cryoprotectant.

In a first embodiment, the invention provides a process for producing a frozen cryopreserving composition comprising biological material, the process comprising the step:
(a) freezing biological material in a cryopreserving composition at a cryopreserving temperature,
   wherein the cryopreserving composition comprises:
(i) an aqueous solution; and
(ii) ice-nucleating agents obtained or obtainable from pollen or spores.

Preferably, the cryopreserving composition additionally comprises:
(iii) a cryoprotectant, preferably DMSO.

In another embodiment, the invention provides a method for improving cell recovery from biological material which has been cryopreserved wherein the biological material comprises cells, the method comprising the steps:
(a) storing the biological material in a cryopreserving composition at a cryopreserving temperature, and optionally
(b) thawing the biological material,
   wherein the cryopreserving composition comprises:
(i) an aqueous solution; and
(ii) ice-nucleating agents obtained or obtainable from pollen or spores.

Preferably, the cryopreserving composition additionally comprises:
(iii) a cryoprotectant, preferably DMSO.

In yet a further embodiment, the invention provides a cryopreserving composition comprising:
(i) ice-nucleating agents obtained or obtainable from pollen or spores; and
(ii) a cryoprotectant.

Preferably, the cryoprotectant is DMSO.

The invention further provides a kit comprising:
(i) ice-nucleating agents obtained or obtainable from pollen or spores; and
(ii) a cryoprotectant; and optionally
(iii) instructions for use of (i) and (ii) in a cryopreservation process or method of the invention.

In yet a further embodiment, the invention provides a composition comprising:
(i) biological material;
(ii) an aqueous solution;
(iii) ice-nucleating agents obtained or obtainable from pollen or spores; and optionally
(iv) a cryoprotectant.

As used herein, the terms “cryopreserving” or “cryopreservation” refer to the storage of biological material, e.g. cells, tissues or organs, as well as viruses, proteins and nucleic acids, at temperatures below 4° C. Generally, the intention of the cryopreservation is to maintain the biological material in a preserved or dormant state, after which time the biological material may be returned to a temperature above 4° C. for subsequent use.

As used herein, the term “biological material” includes, but is not limited to, cell-containing biological material. The term includes cells, tissues, spheroids, organoids, whole organs and parts of organs. In some embodiments, the biological material comprises a cell or cells; or the biological material comprises spheroids.

The cells may be prokaryotic or eukaryotic cells. For example, the cells may be bacterial cells, fungal cells, plant cells, animal cells, preferably mammalian cells, and most preferably human cells.

In some embodiments of the invention, the cells are all of the same type. For example, they are all blood cells, brain cells, muscle cells or heart cells. In other embodiments, the biological material comprises a mixture of one or more types of cell. For example, the biological material may comprise a primary culture of cells, a heterogeneous mixture of cells, organoids or spheroids (preferably spheroids). In other embodiments, the cells are all from the same lineage, e.g. all haematopoietic precursor cells.

The cells for cryopreservation are generally live or viable cells, or substantially all of the cells are live or viable prior to cryopreservation. The biological material may be living or dead (i.e. non-viable) material.

In some embodiments, the cells are ones that grow or are frozen as monolayers (e.g. A549 cells), i.e. the cells are monolayers. In other embodiments, the cells are ones that grow or are frozen in suspension (e.g. Jurkat cells).

In some embodiments, the cells are isolated cells, i.e. the cells are not connected in the form of a tissue or organ.

In some preferred embodiments, the cells are adipocytes, astrocytes, blood cells, blood-derived cells, bone marrow cells, bone osteosarcoma cells, brain astrocytoma cells, breast cancer cells, cardiac myocytes, cerebellar granule cells, chondrocytes, corneal cells, dermal papilla cells, embryonal carcinoma cells, embryo kidney cells, endothelial cells, epithelial cells, erythroleukaemic lymphoblasts, fibroblasts, foetal cells, germinal matrix cells, hepatocytes, intestinal cells, keratocytes, kidney cells, liver cells, lung cells, lymphoblasts, melanocytes, mesangial cells, meningeal cells, mesenchymal stem cells, microglial cells, neural cells, neural stem cells, neuroblastoma cells, oligodendrocytes, oligodendroglioma cells, oocytes, oral keratinocytes, organ culture cells, osteoblasts, ovarian tumour cells, pancreatic beta cells, pericytes, perineurial cells, root sheath cells, schwann cells, skeletal muscle cells, smooth muscle cells, sperm cells, stellate cells, synoviocytes, thyroid carcinoma cells, villous trophoblast cells, yolk sac carcinoma cells, oocytes, sperm or embryoid bodies; or any combination of the above.

In other embodiments, the cells are stem cells, for example, mesenchymal stem cells, neural stem cells, adult stem cells, iPS cells or embryonic stem cells.

In some preferred embodiments, the cells are blood cells, e.g. red blood cells, white blood cells or blood platelets. In some particularly preferred embodiments, the cells are red blood cells which are substantially free from white blood cells and/or blood platelets.

In other embodiments, the biological material to be cryopreserved is in the form of a tissue or a whole organ or part of an organ. The tissues and/or organs and/or parts may or may not be submerged, bathed in or perfused with the composition prior to cryopreservation. Examples of tissues include skin grafts, corneas, ova, germinal vesicles, or sections of arteries or veins. Examples of organs include the liver, heart, kidney, lung, spleen, pancreas, or parts or sections thereof. These may be of human or non-human (e.g. non-human mammalian) origin.

In some preferred embodiments, the biological material or cells are selected from semen, blood cells (e.g. donor blood cells or umbilical cord blood, preferably human), stem cells, tissue samples (e.g. from tumours and histological cross sections), skin grafts, oocytes (e.g. human oocytes), embryos (e.g. those that are 2, 4 or 8 cells when frozen), ovarian tissue (preferably human ovarian tissue) or plant seeds or shoots.

As used herein, the term “biological material” also includes non-cell-containing biological material, e.g. polypeptides and nucleic acids. The nucleic acid may be DNA, e.g. genomic DNA, cDNA, plasmid DNA or vector DNA. The nucleic acid may be RNA, e.g. mRNA. The biological material may also be a virus or viral vector. The polypeptides and/or nucleic acids may be in isolated form, i.e. free or substantially free from other polypeptides and/or nucleic acids, or cells. In some embodiments, the biological material is a vaccine, e.g. a polypeptide vaccine, a DNA vaccine or mRNA vaccine.

The biological material may be a sample which is obtained or which has previously been obtained from a subject. As used herein, the term “subject” can refer to any prokaryotic or eukaryotic organism (e.g. human).

The cryopreserving composition generally comprises an aqueous solution. The aqueous solution may be any aqueous solution which is suitable for the cryopreservation and storage of biological material. Examples include water, saline solution, cell culture media, and physiologically-acceptable buffers.

The cryopreserving composition also comprises: (ii) ice-nucleating agents obtained or obtainable from pollen or spores.

As used herein, the term “ice-nucleating agents” refers to one or more different types of moiety which have ice-nucleating properties. In particular, the ice-nucleating agents will be ones which are capable of inducing ice nucleation in a super-cooled liquid (e.g. water) at a higher temperature compared to a control super-cooled liquid (e.g. water) which does not comprise the ice-nucleating agents.

In some embodiments, the presence of the ice-nucleating agents in the cryopreserving composition raises the ice nucleating temperature at least 2° C., preferably at least 3, 4, or 5° C. (compared to a control cryopreserving composition without the ice-nucleating agents). In other embodiments, the presence of the ice-nucleating agents in the cryopreserving composition raises the ice nucleating temperature to at least −10° C., preferably at least −9° C., −8° C., −7° C. or −6° C.

The ice-nucleating agents are obtained or are obtainable from pollen or spores. The pollen is plant pollen; the spores are plants spores. In some embodiments, the pollen is tree pollen. The plant may, for example, be an angiosperm or a gymnosperm.

TABLE 1
List of plant pollens tested for ice nucleation. (Ice nucleation data is given in FIG. 5).

| | Common | | | |
|---|---|---|---|---|
| Species | Name | Genus | Family | Order |
| Sambucus nigra | elderberry | Sambucus | Adoxaceae | Dipsacales |
| Various (to be | daffodil | Narcissus | Amaryllidaceae | Asparagales |
| identified) | | | | |
| Hymenocallis littoralis | beach spider | Hymenocallis | Amaryllidaceae | Asparagales |
| | lily | | | |
| Araucaria araucana | monkey puzzle | Araucaria | Araucariaceae | Pinales |
| Hyacinthus orientalis | common | Hyacinthus | Asparagaceae | Asparagales |
| | hyacinth | | | |
| Betula pendula | silver birch | Betula | Betulaceae | Fagales |
| Carpinus betulus | European | Carpinus | Betulaceae | Fagales |
| | hornbeam | | | |
| Corylus avellana | common hazel | Corylus | Betulaceae | Fagales |
| Alnus glutinosa | black alder | Alnus | Betulaceae | Fagales |
| Cistus populifoloius | rock rose | Cistus | Cistaceae | Malvales |
| Sequoiadendron | wellingtonia | Sequoia- | Cupressaceae | Pinales |
| giganteum | | dendron | | |
| Cupressus | Mediterranean | Cupressus | Cupressaceae | Pinales |
| sempervirens | cypress | | | |
| Erica multiflora | many-flowered | Erica | Ericaceae | Ericales |
| | heath | | | |
| Crocus vernus | Dutch crocus | Crocus | Iridaceae | Asparagales |
| Clerodendrum | Java | Clerodendrum | Lamiaceae | Lamiales |
| speciosissimum | glorybower | | | |
| Fritillaria meleagris | snake's head | Fritillaria | Liliaceae | Liliales |
| | fritillary | | | |
| Musa rubra | red dwarf | Musa | Musaceae | Zingiberales |
| | banana | | | |
| Nymphaea ‘Kew's | water lily | Nymphaea | Nymphaeaceae | Nymphaeales |
| Stowaway Blues’ | | | | |
| hybrid | | | | |
| Pinus halepensis | Aleppo pine | Pinus | Pinaceae | Pinales |
| Pinus coulteri | coulter pine | Pinus | Pinaceae | Pinales |
| Pinus mugo | dwarf | Pinus | Pinaceae | Pinales |
| | mountain pine | | | |
| Picea brachytyla | sargent spruce | Picea | Pinaceae | Pinales |
| Picea wilsonii | Wilson's | Picea | Pinaceae | Pinales |
| | spruce | | | |
| Plantago lanceolata | ribwort plantain | Plantago | Plantaginaceae | Lamiales |
| Cynosurus cristatus | crested dog's- | Cynosurus | Poaceae | Poales |
| | tail | | | |
| Dactylis glomerata | cat grass | Dactylis | Poaceae | Poales |
| Arundo formosana | Taiwan grass | Arundo | Poaceae | Poales |
| Aesculus | horse chestnut | Aesculus | Sapindaceae | Sapindales |
| hippocastanum | | | | |
| Encephalartos | Ugandan | Encephalartos | Zamiaceae | Cycadales |
| equatoralis | cycad | | | |
| Hedychium | butterfly lily | Hedychium | Zingiberaceae | Zingiberales |
| coronarium | | | | |

In some embodiments, the pollen is from a plant from an Order listed in Table 1. In some embodiments, the pollen is from a plant from a Family listed in Table 1. In some embodiments, the pollen is from a plant from a Genus listed in Table 1.

In some embodiments, the pollen is from a plant of the Order Amborellales, Nymphaeales, Austrobaileyales, Magnoliales, Laurales, Piperales, Canellales, Chloranthales, Arecales, Poales, Commelinales, Zingiberales, Asparagales, Liliales, Diosco reales, Pandanales, Petrosaviales, Alismatales, Acorales, Ceratophyllales, Ranunculales, Proteales, Trochodendrales, Buxales, Gunnerales, Fabales, Rosales, Fagales, Cucurbitales, Oxalidales, Malpighiales, Celastrales, Zygophyllales, Geraniales, Myrtales, Crossosomatales, Picramniales, Malvales, Brassicales, Huerteales, Sapindales, Vitales, Saxifragales, Dilleniales, Berberidopsidales, Santalales, Caryophyllales, Cornales, Ericales, Aquifoliales, Asterales, Escalloniales, Bruniales, Apiales, Dipsacales, Paracryphiales, Solanales, Lamiales, Vahliales, Gentianales, Boraginales, Garryales, Metteniusales or Icacinales.

In other embodiments, the pollen is from a plant of the Order Cycadales, Ginkgoales, Welwitschiales, Gnetales, EphedralesOrder Pinales, Araucariales or Cupressales.

Preferably, the pollen is from Sambucus nigra, Hymenocallis littoralis, Araucaria Araucana, Hyacinthus orientalis, Betula pendula, Carpinus betulus, Corylus avellana, Alnus glutinosa, Cistus populifoloius, Sequoiadendron giganteum, Cupressus sempervirens, Erica multiflora, Crocus vernus, Clerodendrum speciosissimum, Fritillaria meleagris, Musa rubra, Nymphaea ‘Kew's Stowaway Blues’, Pinus halepensis, Pinus coulteri, Pinus mugo, Picea brachytyla, Picea wilsonii, Plantago lanceolata, Cynosurus cristatus, Dactylis glomerata, Arundo formosana, Aesculus hippocastanum, Encephalartos equatoralis, or Hedychium coronarium.

Most preferably, the tree pollen is from silver birch (Betula pendula), European hornbeam (Carpinus betulus) or dwarf mountain pine (Pinus mugo).

Pollen from these trees (and many others) may be purchased from Pharmallerga (Czech Republic).

In other embodiments, the pollen is non-tree pollen. Examples of non-tree pollen include pollen from stinging nettle.

In some embodiments, the spores are fern (Polypodiopsida or Polypodiophyta) spores.

As used herein, the term “pollen” includes pollen from a single species of plant or a mixture of pollens from more than one species of plant.

The ice-nucleating agents (e.g. polysaccharides) are water dispersible. In some embodiments, the ice nucleating agents (e.g. polysaccharides) are water-soluble.

In some embodiments, the ice nucleating agents (e.g. polysaccharides) are soluble at temperatures above 0° C. In some embodiments, the ice nucleating agents (e.g. polysaccharides) are soluble at temperatures below 0° C. In other embodiments, the ice nucleating agents (e.g. polysaccharides) are not soluble at temperatures below 0° C.

In some preferred embodiments, the ice-nucleating agents obtained or obtainable from the pollen or spores is provided in the form of an infusion (preferably an aqueous infusion) which is or has been obtained from the pollen or spores, preferably pollen washing water (PWW). As used herein, the term “pollen washing water (PWW)” relates to an infusion which has been obtained after allowing a defined amount or number of pollen grains to be suspended in a defined volume of solvent for a defined time period, and then the (insoluble) pollen grains are removed from the solvent (e.g. by filtration). The infusion is the resultant liquid. Examples of suitable solvents include water-soluble solvents or aqueous solvents, such as water, cell culture media, DMSO, glycerol, trehalose solutions and propylene glycol. The infusion is preferably an aqueous infusion.

The amount of ice-nucleating agents which is required to be present in the cryopreserving composition may vary between compositions and the biological material to be cryopreserved. However, the skilled person will readily be able to determine this amount following the teaching herein, combined with the skilled person's common general knowledge.

In some embodiments, the amount of the ice-nucleating agents in the cryopreserving composition is an amount which is sufficient to raise the ice nucleating temperature by at least 2° C., preferably at least 3, 4, 5, 6, 7 or 8° C. (compared to a control cryopreserving composition without the ice-nucleating agents).

In other embodiments, the amount of the ice-nucleating agents in the cryopreserving composition is an amount wherein the ice nucleating temperature in the cryopreserving composition is at least −10° C., preferably at least −9° C., −8° C., −7° C. or −6° C.

In the production of the infusion (e.g. PWW), the defined amount of pollen grains to be suspended in a defined volume of solvent may, for example, be 0.00001 g/L to 1000 g/L. In some embodiments, the defined amount is 0.00001 g/L to 0.0001 g/L, 0.0001 g/L to 0.001 g/L, 0.001 g/L to 0.01 g/L, 0.01 g/L to 0.1 g/L, 0.1 g/L to 1 g/L, 1 g/L to 10 g/L 10 g-100 g/L, or 100 g-1000 g/L. In some preferred embodiments, the defined amount is 0.2 g/10 ml or 20 g pollen/litre solvent (preferably water).

In other embodiments, in the production of the infusion (e.g. PWW), the defined number of pollen grains is 1×10³ to 1×10¹¹ pollen grains/litre of solvent, e.g. 1×10³-1×10⁴, 1×10⁴-1×10⁵, 1×10⁵-1×10⁶, 1×10⁶-1×10⁷, 1×10⁷-1×10³, 1×10¹-1×10⁹, 1×10⁹-1×10¹⁰ or 1×10¹⁰-1×10¹¹, pollen grains/litre of solvent. For example, 1 million Betula pendula pollen grains/L may be used.

In the production of the infusion (e.g. PWW), the defined time period may be at least 1-5 minutes, 5-30 minutes, 30-60 minutes, or 1, 2, 5, 10 or 20 hours, e.g. 12-36 hours or about 24 hours.

In another example, a suitable pollen washing water (PWW) may be prepared by suspending pollen grains in a suitable cryoprotectant (for example, a DMSO-based cryoprotectant) or water as shown in FIG. 1.

The infusion process may be performed at any suitable temperature. Such temperatures include ambient temperature and 4° C.

In some embodiments, the mass concentration of the ice-nucleating agents in the infusion (e.g. PWW) is 6.0-8.0 mg/ml, e.g. about 6.6 mg/mL or about 7.0 mg/mL.

The pollen grains may be removed from the infusion by filtration or any other suitable means (e.g. centrifugation). The filter size will be dependent on the size of the pollen being used. The smallest pollen grains are about 5 μm in diameter. Preferably, the filter pore size is in the range of 3 nm to 150 μm. In some preferred embodiments, the pore size is about 0.2 μm.

Hence the ice-nucleating agents will be ones which are capable of passing through a filter with a maximum pore size of 150 μm. In some embodiments, the ice-nucleating agents are ones which are capable of passing through a filter with a maximum pore size of 100 μm, 50 μm, 10 μm, 1 μm, 500 nm, 100 nm, 50 nm or 10 nm. In some preferred embodiments, the ice-nucleating agents are ones which are capable of passing through a filter with a maximum pore size of 200 nm.

The PWW may be used in the form of the (liquid) infusion or a desiccated extract thereof.

In some embodiments, the cryopreserving composition consists of or comprises the infusion (e.g. PWW) or 1-99% (v/v) infusion, optionally together with a cryoprotectant (e.g. DMSO). In some embodiments, the cryopreserving composition comprises 20-80% infusion, 30-70%, 40-60% or about 50% infusion (e.g. PWW).

In other embodiments, the cryopreserving composition comprises ice nucleating agents which have been obtained from a defined number of pollen grains. For example, the cryopreserving composition may comprise ice nucleating agents which have been obtained from 5,000 to 20,000 pollen grains or from about 10,000 pollen grains. In particular, the cryopreserving composition may comprise ice nucleating agents which have been obtained from about 10,000 Betula pendula or Carpinus betulus pollen grains. In particular, the cryopreserving composition may comprise ice nucleating agents which have been obtained from about 1,000-2,000 Pinus mugo pollen grains.

In some embodiments, the ice-nucleating agents obtained or obtainable from pollen or spores are or include water-soluble polysaccharides or water-soluble polysaccharides bearing carboxylated groups, which are obtained or obtainable from pollen or spores.

For example, the ice-nucleating agents obtained or obtainable from pollen or spores may be a polysaccharide-enriched and/or monosaccharide-enriched fraction obtained from an infusion from the pollen or spores.

In some embodiments, the polysaccharide fraction has a molecular weight of 50-400 kDa, e.g. 100-300 kDa.

For example, the polysaccharide/monosaccharide fraction may be one which is capable of passing through a filter with a maximum pore size of 150 μm. In some embodiments, the polysaccharide/monosaccharide fraction is one which is capable of passing through a filter with a maximum pore size of 100 μm, 50 μm, 10 μm, 1 μm, 500 nm, 100 nm, 50 nm or 10 nm. In some preferred embodiment, the polysaccharide/monosaccharide fraction is one which is capable of passing through a filter with a maximum pore size of 200 nm

The polysaccharides in the polysaccharide fraction (and/or monosaccharides) may be provided in solution (i.e. in the infusion) or in desiccated form.

The cryopreserving composition may additionally comprise a cryoprotectant. As used herein, the term “cryoprotectant” relates to a substance used to help protect biological tissue (e.g. cells) from freezing damage (i.e. damage due to ice formation). Examples of conventional cryoprotectants include, but are not limited to, dimethyl sulfoxide (DMSO), ethylene glycol, glycerol-2-methyl-2,4-pentanediol (MPD), propylene glycol, sucrose and trehalose.

In preferred embodiments, the cryoprotectant is DMSO. In the cryopreserving compositions, the DMSO may be at a concentration of 1%-20% (v/v), preferably 5%-15%, and more preferably about 10%.

The cryopreserving composition may additionally comprise one or more of the following: a buffer (e.g. PBS, FBS, cell media), an antibiotic, an anticoagulant, an antioxidant, and a pH indicator.

The composition preferably does not contain haemolytic agents, e.g. agents which induce the lysis of red blood cells.

The biological material may be cryopreserved in any suitable container.

In some embodiments, the biological material is cryopreserved in individual compartments of a well plate. As used herein, the term “well plate” refers to a plate (e.g. a microtitre well plate) comprising a plurality of discrete compartments for holding samples and/or reagents. Such well plates may comprise any number of discrete compartments. In certain embodiments, the well plate is a 48-well or 96-well plate. Plates with more wells, e.g. a 384-well plate, are also available. The well plate is preferably made of plastic, e.g. polyethylene, polypropylene or polystyrene, preferably polystyrene.

In some embodiments, the volume of the cryopreserving composition (including the biological material) to be frozen is 1 μl to 1 ml, or 1 ml to 1 litre. For example, the volume may be 1-10 μl, 10-50 μl, 50-100 μl, 100-500 μl, 500-1000 μl, 1-10 ml, 10-100 ml, 100-500 ml or 500-1000 ml. In other examples, the volume is 1-2.5 ml (e.g. a cryovial or Eppendorf tube).

In some preferred embodiments, the step of freezing the biological material in a cryopreserving composition at a cryopreserving temperature is performed when the cryopreserving composition is present in one or more wells of a 48-, 96- or 384-well (microtitre) plate.

In some preferred embodiments, the total volume of cryopreserving composition is 25-400 μl, more preferably 50-100 μl or 100-200 μl or 200-400 μl, and most preferably 75-125 μl or about 50 μl or about 100 μl. The volumes may be used in the wells of a well plate; they may also be used in other containers.

The biological material is contacted with the cryopreserving composition. In general, the biological material will be immersed or submerged in the cryopreserving composition or perfused with the cryopreserving composition such that the cryopreserving composition makes intimate contact with all or with substantially all of the biological material before the start of the freezing step. In general, the biological material will be contacted with the cryopreserving composition in this way before the temperature is reduced, i.e. before the start of the freezing step.

In general, the cryopreserving composition comprising the biological material will initially be at a temperature above 0° C., e.g. at about 4° C. or at ambient temperature. From there, its temperature will be reduced to the cryopreserving temperature, preferably in a single, essentially uniform step (i.e. without a significant break). Alternatively, the temperature may first be reduced to an intermediate temperature (which may be above or below the final cryopreserving temperature).

As used herein, the term “freezing” or “frozen” refers to reducing the temperature to a cryopreserving temperature or being at a cryopreserving temperature. The rate of this freezing step is preferably slow (e.g. 0.5-50° C./minute). In some embodiments, the rate is 0.5-1.5° C./minute or about 1° C./minute.

The most preferred freezing rate in any one particular case will be dependent on the volume of the cryopreserving composition and the nature of the biological material. By following the teachings herein and the above points in particular, the skilled person may readily determine the most appropriate freezing rate in any one case.

As used herein, the term “ice nucleation” refers to the process by which the molecules in a liquid gather into clusters, arranging in a way that will eventually define the crystal structure of the solid. In simple terms, the term “ice nucleation” refers to the formation of ice.

The temperature at which ice nucleation of the biological material occurs may be below 0° C., below −5° C. or below −10° C. In some embodiments, ice nucleation occurs at a temperature between −10° C. and 0° C., preferably between −9° C. to −5° C. and most preferably between −8° C. and −6° C. Ice nucleation within the cryopreserving composition may be tested for by differential scanning calorimetry or cryomicroscopy.

The invention therefore provides a process as described herein, wherein ice is present in the cryopreserving composition at one or more stages during thawing of the composition.

The cryopreserved material may be stored at the cryopreserving temperature for any desired amount of time. Preferably, the cryopreserved biological material is stored for at least one day, at least one week or at least one year. More preferably, it is stored for 1-50 days, 1-12 months or 1-4 years. In some embodiments, it is stored for less than 5 years.

The cryopreserved biological material may be stored for use in, for example, cell banking. The cryopreserved biological material may be stored for cell, tissue and/or organ banking.

The processes and methods of the invention may additionally comprise the step of thawing the cryopreserving composition. As used herein, the term “thawing” refers to raising the temperature of the cryopreserved composition or biological material to 0° C. or above, preferably to 4° C. or above. In some embodiments, the term “thawing” refers to raising the temperature of the composition or biological material to a temperature at which there are no or substantially no ice crystals in all or part of the composition or biological material. Hence the term “thawing” includes complete and partial thawing.

The rate of thawing may, for example, be slow (e.g. 1-10° C./minute) or fast (above 10° C./min). In some cases it may be advantageous to thaw slowly. Rapid thawing in a water bath at 37° C. is preferred. Cell recovery is also possible at lower temperatures (e.g. 20° C.).

After cryopreservation and subsequent thawing, the biological material may be isolated from the cryopreserving composition and/or used for any desired use. The processes and methods of the present invention may therefore further comprise a step of isolating or removing the biological material from the cryopreserving composition. Desired uses may include human and veterinary uses, tissue engineering, gene therapy and cellular implantation.

Preferably, the process/method steps are carried out in the order specified.

In other embodiments, the invention provides a method for reducing cell damage during the freezing of biological material, wherein the biological material comprises cells. Cell damage may be assessed based on the viability of the cells.

In certain embodiments, the cells or substantially all of the cells in the biological material are cryopreserved (i.e. frozen) in a viable state. It is of interest to ensure such cells remain viable after cryopreservation, thawing and subsequent obtainment.

Therefore, the invention provides a means by which the cell viability of the biological material is enhanced after the cryopreservation procedure compared to a method which does not use ice-nucleating agents obtained or obtainable from tree pollen, as defined herein.

The cell viability of a biological material that has been cryopreserved can be assessed by a number of methods known in the art. For example, the metabolic activity of cells of the biological material can be assessed post-thawing.

In yet a further embodiment, the invention provides a cryopreserving composition comprising:
(i) ice-nucleating agents obtained or obtainable from pollen or spores; and
(ii) a cryoprotectant.

Preferably, the cryoprotectant is DMSO. The cryopreserving composition may also comprise an aqueous solution, e.g. as defined herein. The cryoprotectant (e.g. DMSO) may be at a concentration of 1%-20% (v/v), preferably 5%-15%, and more preferably about 10%.

The cryopreserving composition may additionally comprise (iii) biological material.

In some embodiments, the cryopreserving composition is a cryopreserved composition, i.e. it is frozen at a cryopreserving temperature.

The invention further provides a kit comprising:
(i) ice-nucleating agents obtained or obtainable from pollen or spores; and
(ii) a cryoprotectant; and optionally
(iii) instructions for use of (i) and (ii) in a cryopreservation process or method of the invention.

The cryopreserving composition or kit may additionally comprise one or more of the following: a buffer (e.g. PBS), an antibiotic, an anticoagulant, an antioxidant and/or a pH indicator.

The disclosure of each reference set forth herein is specifically incorporated herein by reference in its entirety.

### BRIEF DESCRIPTION OF THE FIGURES

FIG. 1 shows a schematic diagram of a method for preparing pollen washing water (PWW).

FIG. 2 shows measurements of the freezing temperature of birch and hornbeam PWWs.
(a) Plot of droplet fraction frozen as a function of temperature for pure water and PWWs in various volumes, including measurements from Daily et al.³ for pure water. The shaded blocks represent estimated freezing ranges for 100 μl volumes of MilliQ® water and PWWs from measurements made with embedded thermocouples.
(b) Plot of cumulative number of ice nucleators per pollen grain for literature data from Dreischmeier et al.³⁰ and measurements made in this study on birch and hornbeam PWW.
(c) Temperature trace from thermocouples embedded in 96-well plates containing 92 100 μl volumes of the two PWWs and MilliQ® water.

FIG. 3 shows a schematic diagram of a cryopreservation procedure used to measure the effectiveness of PWW as a cryoprotectant. The micrographs show A549 cell monolayers before cryopreservation and after cryopreservation, with and without use of PWW. The scale bars on the micrographs represent 100 μm.

FIG. 4 shows cryopreservation enhancement in 96-well plates. (a-f) Boxplots of metabolic activity as measured by resazurin assay 24 hours post-thaw for individual batches of A549 and Jurkat cells. The number of wells measured for each experiment can be found in Table 3. (b) Average post-thaw metabolic activity for Jurkat cells. (c) Average post-thaw metabolic activity for A549 cells. For panels (b) and (c), the error bars are the standard error of the mean, while the P-values are 2-sample t-tests comparing the with-PWW and without-PWW conditions for each cell type and cooling rate.

FIG. 5 shows a plot of data for freezing of microlitre droplets containing pollen washing water (PWW) from the plants listed in Table 1. PWW from some pollens (e.g. Pinus mugo Hymenocalis littoralis, and Sequoiadendron giganteum) work at much warmer temperatures than Carpinus betulus. It is likely that these latter pollens will enhance cryopreservation to a greater extent than Carpinus betulus, particularly in procedures that aim to cryopreserve smaller volumes of biological material.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The following Materials and Methods were used in one or more of the Examples.

### Preparation of Pollen Washing Water (PWW)

To produce PWW for ice nucleation experiments, 0.2 g of pollen from either silver birch (Betula pendula) or European hornbeam (Carpinus betulus), both purchased from Pharmallerga, was suspended in 10 mL MilliQ® water overnight, then filtered through a 0.2 μm filter. This procedure produces a transparent yellow liquid free from pollen grains. For cell cryopreservation experiments, PWW was prepared as described using complete cell culture media in place of water.

### Conversion Between Pollen Mass Per Litre of Water and Number of Pollen Grains Per Litre of Water

Table 2 (below) shows the conversion between pollen mass per litre of water and number of pollen grains per litre of water, for Carpinus betulus (European Hornbeam). Other pollens have different densities and grain sizes. Approximations regarding sphericity, grain-size consistency, etc., have been made. Line A is the concentration used herein for ice nucleation experiments; Line B is the concentration used for cryopreservation procedures.

Ice nucleation activity is due to the total amount of nucleating material in a given volume of water. Hence for nucleation to occur, on average, at −14° C. in a 1 μl droplet you need the soluble material from about 3,000 Carpinus betulus pollen grains. To get an average nucleation temperature of −7° C., droplets are needed which are 100 times larger and which, hence, contain the material from 30,000 pollen grains. For Pinus mugo, 100 times smaller droplets may be used to achieve the same nucleation temperature as in 1 μl droplets of Carpinus betulus. As such, one can expect to need to use at least 100 times less material for the same effect in 100 μl scale cryopreservation procedures (i.e. circa 3000 grains per 100 μl droplet).

TABLE 2
Conversion between pollen mass per litre of water and number
of pollen grains per litre of water for Carpinus betulus.

| | Pollen | Mean | Mean | Mean |
|---|---|---|---|---|
| | mass per | number of | number of | number of |
| | litre | pollen | pollen | pollen |
| | of water | grains per | grains per | grains per |
| Line | (g/l) | litre | millilitre | microlitre |
| | 0.00001 | 1381.55 | 1.38 | 0.00 |
| | 0.0001 | 13815.53 | 13.82 | 0.01 |
| | 0.001 | 138155.33 | 138.16 | 0.14 |
| | 0.01 | 1.38E+06 | 1381.55 | 1.38 |
| | 0.1 | 1.38E+07 | 13815.53 | 13.82 |
| | 1 | 1.38E+08 | 138155.33 | 138.16 |
| B | 10 | 1.38E+09 | 1.38E+06 | 1381.55 |
| A | 20 | 2.76E+09 | 2.76E+06 | 2763.11 |
| | 100 | 1.38E+10 | 1.38E+07 | 13815.53 |
| | 500 | 6.91E+10 | 6.91E+07 | 69077.67 |

### Measurement of Ice Nucleation Temperatures

To determine the freezing temperatures of microlitre droplets, a droplet freezing assay based on that described by Whale et al.³⁵ was used. Arrays of approximately 40 one microlitre droplets were pipetted onto silanised glass slides (Hampton Research HR3-231) and cooled using an aluminium Peltier-driven cold stage. The Peltier was controlled by a Meerstetter TEC-1091 reading a Netshushin 1 mm diameter PT-100 embedded directly under the glass slide. A recirculating chiller was used as backing cooling for the cold stage. Separately, a PicoTech PT-104 was used to monitor two more PT100s embedded in different locations under the glass slide. A camera monitored the freezing of the droplets, allowing the fraction of droplets frozen at a given temperature to be determined.

To investigate nucleation temperatures directly in standard flat-bottom Society for Biomolecular Sciences flat-bottom 96-well plates (Corning), a Cytiva Viafreeze Uno (VFU) controlled rate freezer was used to cool plates containing 92 100 μL aliquots of MilliQ® water, birch PWW and hornbeam PWW at a nominal rate of 2° C./min. T-type thermocouples were embedded in the remaining 4 wells using pressure sensitive putty. The wells containing thermcouples were central to the four quadrants of the plate. The thermocouples were calibrated against Netshushin PT-100s. A thermocouple logger integrated into the VFU was used to monitor the temperature of the plates during cooling. Freezing was detected by deviation of the steady temperature ramp caused by the release of latent heat during water freezing as shown in FIG. 2. (a). The same method was used to monitor freezing temperatures and cooling rates during a subset of the cryopreservation procedures also. It should be noted that the deviation from the initial steady cooling rate is associated with freezing, as opposed to nucleation; and that there will be a degree of lag between the nucleation event and detection of resultant latent heat associated with the thermal conductivity of the plate and the putty used. It is anticipated that at cooling rates of 0.5° C./min and 2° C./min, these effects are quite small but they may lead to an overestimate of the degree of supercooling reached before nucleation.

### Cell Cultures

Human Caucasian lung carcinoma cells (A549) (ECACC 86012804) and Jurkat E6.1 cells (ECACC 88042803) were obtained from the European Collection of Authenticated Cell Cultures, and cultured in complete culture media consisting of F-12K (Gibco) or Advanced RPMI 1640 (Gibco), respectively, containing 10% (v/v) fetal bovine serum (FBS) (Merck), 100 units·mL-1 penicillin, 100 μg·mL-1 streptomycin and 250 ng·mL-1 amphotericin B (Gibco). Cells were maintained at 37° C., 5% CO₂ and sub-cultured every 3-4 days.

### Cryopreservation of A549 Cells in 96-Well Plates

Adherent A549 monolayers were washed with sterile phosphate buffered saline (PBS) then dissociated using 0.25% trypsin plus 1 mM ethylenediaminetetraacetic acid (EDTA) in balanced salt solution. Following centrifugation at 180×g for 5 minutes, a sample of A549 cells was diluted 1:1 in 0.4% trypan blue and counted using a haemocytometer. Cells were resuspended at a cell density of 2×10⁵ cells/mL. 100 μL of cell suspension (2×10⁴ cells/well) was added to individual wells of a flat-bottom 96-well plate (Corning) and cells were allowed to adhere overnight. The following day, cryoprotectant solutions were prepared, consisting of 10% (v/v) DMSO in either 50% (v/v) PWW or complete cell media. Media was removed from wells and 50 μL cryoprotectant was added to each well. Plates were either placed directly in a freezer set to −80° C. for 24 hours or placed in a ViAfreeze Uno controlled rate freezer and frozen at either 0.5 or 2° C./min until the plate reached −80° C. At this point plates were transferred on dry ice to a freezer set to −80° C. for 24 hours.

To thaw, 100 μL of warm (37° C.) complete cell media was added to each well and the plate was incubated at 37° C., 5% CO₂ for 10 minutes. The media was replaced with 100 μL complete media and non-frozen control cells were added to the plate at 1×10⁵ cells/mL in 100 μL. The plate was then incubated for 24 h at 37° C., 5% CO₂. After 24 h the supernatant was removed and 100 μL resazurin solution (prepared in phenol red-free F-12 media containing 10% (v/v) FBS, 100 units·mL⁻¹ penicillin, 100 μg·mL⁻¹ streptomycin and 250 ng·mL⁻¹ amphotericin B) was added to each well. The plate was incubated at 37° C., 5% CO₂ and the absorbance was monitored until 70% reduction was achieved for non-frozen controls.

### Cryopreservation of Jurkat Cells in 96-Well Plates

A sample of Jurkat cells was diluted 1:1 in trypan blue and counted using a haemocytometer. Following counting, cells were centrifuged at 180×g for 5 minutes and resuspended at a cell density of 2×10⁶ cells/mL. 25 μL of cell suspension was added to individual wells of a U-bottom 96-well plate (5×10⁴ cells/well). DMSO (10% (v/v)) was added to solutions of either PWW or complete cell media to prepare cryoprotectant solutions at 2× final concentration. 25 μL of 2× cryoprotectant solution was added to each well and mixed, final PWW concentration 50% (v/v) and 5% DMSO (v/v). Plates were either placed directly in a freezer set to −80° C. for 24 h or placed in a VIAfreeze Uno controlled rate freezer and frozen at either 0.5 or 2° C./min until the plate reached −80° C. At this point plates were transferred on dry ice to a freezer set to −80° C. for 24 h.

To thaw, 100 μL of warm (37° C.) complete cell media was added to each well and the plate was incubated at 37° C., 5% CO₂ for 10 minutes, then centrifuged (730×g for 5 minutes) to pellet cells. The supernatant was removed and cells were resuspended in 100 μL fresh media followed by 24 hours incubation at 37° C., 5% CO₂. After 24 hours, non-frozen controls were prepared at 5×10⁵ cells/well and added to the plate in 100 μL. The plate was centrifuged (730×g for 5 minutes) to pellet cells then the media was replaced with 100 μL resazurin solution (prepared in phenol red-free RPMI 1640 media containing 10% (v/v) fetal bovine serum (FBS), 100 units·mL⁻¹ penicillin, 100 μg·mL⁻¹ streptomycin and 250 ng·mL⁻¹ amphotericin B). The plate was incubated at 37° C., 5% CO₂ and the absorbance monitored until 70% reduction was achieved for non-frozen controls.

### Toxicity of Pollen Washings on A549 Cells and Jurkat Cells

A549 cells were added to a flat-bottom 96-well plate at a density of 1×10⁵ cells/mL (1×10⁴ cells/well) and allowed to adhere overnight. PWW were prepared in complete cell media as previously described. Serial dilutions of PWW were prepared by diluting the stock solution 1:1 in complete cell culture media. Solutions were added at 100 μL/well and the plate was incubated at 37° C., 5% CO₂ for 24 hours. After 24 hours, the supernatant was removed and cell metabolic activity was assessed by the resazurin assay, where absorbance was monitored until 70% reduction was achieved relative to media-only controls.

Jurkat cells were prepared at a cell density of 5×10⁵ cells/mL and 100 μL of cells was added to individual wells of a U-bottom 96-well plate (5×10⁴ cells/well). The plate was centrifuged at 730×g for 5 minutes and the supernatant was removed. PWW were prepared in complete cell media as previously described. Serial dilutions of PWW were prepared by diluting the stock solution 1:1 in complete cell culture media. Solutions were added at 100 μL/well and the plate was incubated at 37° C., 5% CO₂ for 24 hours. After 24 hours, the plate was centrifuged at 730×g, the supernatant was removed and cell metabolic activity was assessed by the resazurin assay, where absorbance was monitored until 70% reduction was achieved relative to media-only controls.

### Live/Dead Assay of A549 Cells

A549 cells were added to a flat-bottom 96-well plate at a density of 1×10⁵ cells/mL (1×10⁵ cells/well) and allowed to adhere overnight. PWW solutions were prepared in complete cell media as previously described. Serial dilutions of PWW were prepared by diluting the stock solution 1:1 in complete cell culture media. PWW solutions were added at 100 μL/well and the plate was incubated at 37° C., 5% CO₂ for 24 hours. After 24 hours, control cells were treated with 70% methanol in deionised water for 15 minutes at 37° C. as a ‘dead’ control. Media was removed from all wells and cells washed with 100 μL PBS. A live/dead solution was prepared by adding 2.5 μM calcein-AM and 15 μM ethidium homodimer-1 to 5 mL sterile PBS and vortexed to mix. 100 μL of the live/dead solution was added to each well and the plate was incubated at room temperature protected from light for 30 min. After 30 min, phase contrast and fluorescence images were captured for each well at 530 and 645 nm on a CKX41 microscope with pE-300-W LED illumination and a XC30 camera. Three wells were analysed for each condition. Image analysis was performed using ImageJ software, version 1.52.

### Statistical Analysis

The relationship between normalised metabolic rate and the two explanatory variables (presence or absence of pollen and freezing rate) was analysed using linear mixed effect models. Linear mixed effect models were fitted by restricted maximum likelihood (REML) and were used to ensure that any variability associated with the three experimental repeats could be appropriately considered in the random effects structure of the model. Stepwise model selection was conducted using ANOVA and Akaike information criterion (AIC). Model fit was assessed via visual inspection of the residuals. Models were fitted separately to each cell line (A549 and Jurkat). All models were fitted in R (R Core Team, 2020; version 4.0.2) using the Ime4 package.45 Data visualisation was conducted in OriginPro 2019b.

### Example 1: Freezing Temperatures of Water and PWW in 96-Well Plates

We used a microlitre droplet freezing assay similar to that described by Whale et al.³⁵ to quantify the ice nucleation ability of two tree pollens. This technique cools an array of approximately 40 one microlitre droplets at a rate of 2° C./min. A camera was used to detect freezing of droplets during cooling, allowing the fraction of droplets frozen as a function of temperature to be determined.

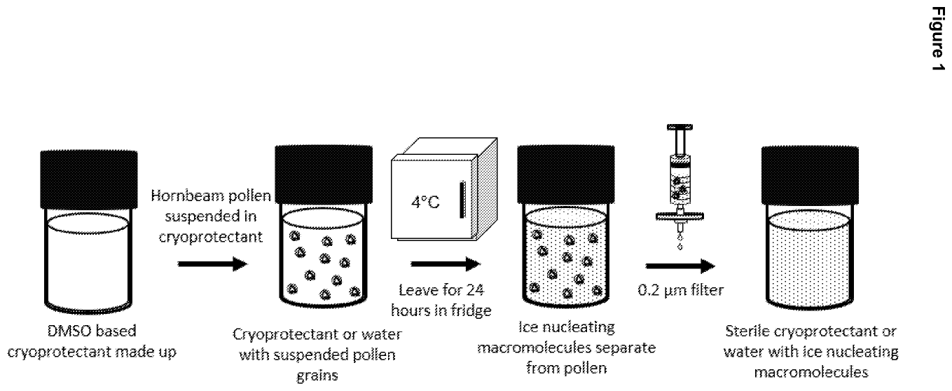

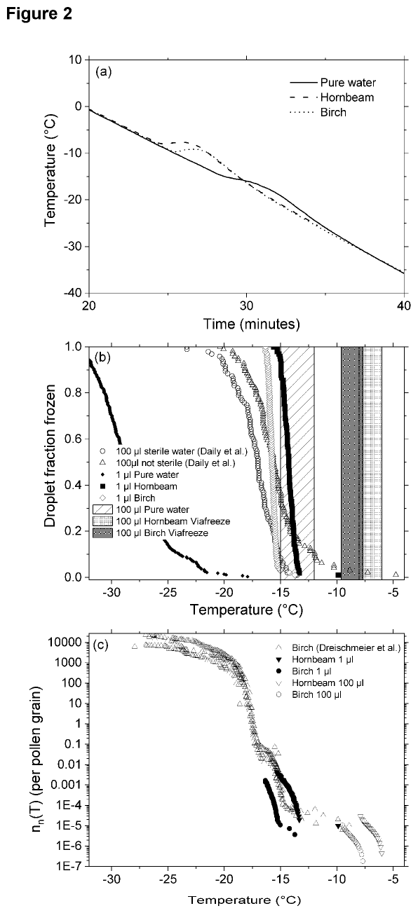

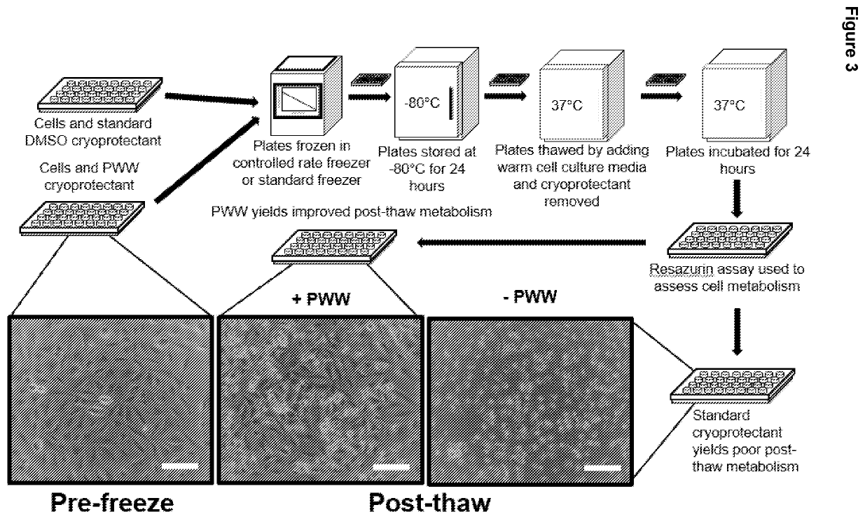

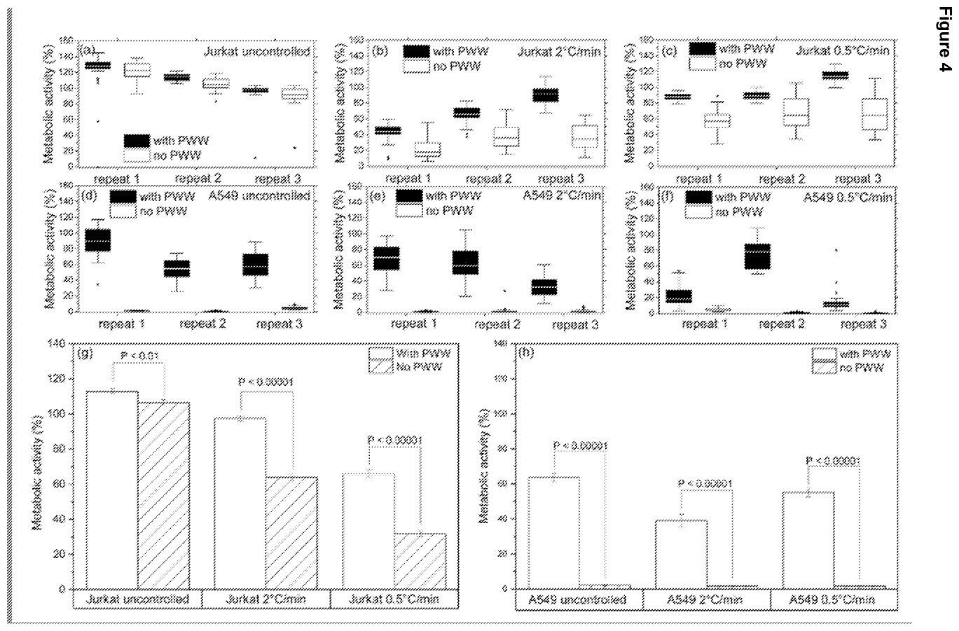

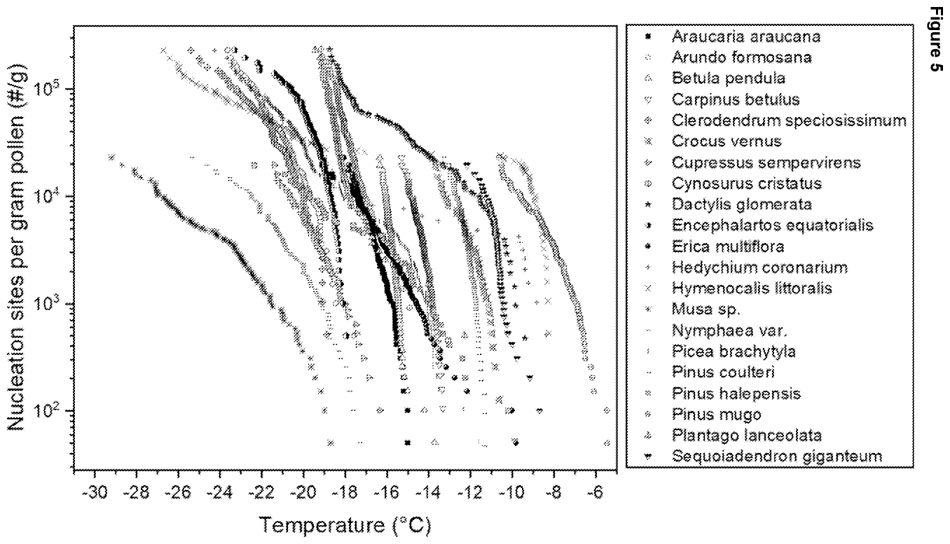

As shown in FIG. 1, PWW was prepared by suspending pollen grains in water or cryoprotectant solution leaving the suspension at 4° C. for 24 hours, then filtering off the pollen grains. We prepared PWWs using 2 wt % of pollen from silver birch (Betula pendula) and European hornbeam (Carpinus betulus). Both pollens are readily available from commercial suppliers. Experiments to determine nucleation temperatures were conducted on PWWs prepared in pure MilliQ® water, rather than cryoprotectant solutions.

The results of these experiments are shown in FIG. 2(b). We found that birch PWW induced freezing at an average temperature of −15.1° C., while PWW from hornbeam induced freezing at an average temperature of −14.5° C.

To directly compare our data with literature measurements made by Dreischmeier et al.¹ we calculated the cumulative number of ice nucleators per pollen grain on cooling from 0° C. to temperature T, called nₙ(T), using the equation¹:

$${n}_{n} ( T ) = \frac{- ln ( 1 - f ( T ) )}{n}$$

where f is the droplet fraction frozen at temperature T and n is the number of pollen grains which contributed to the PWW in each droplet. This is a non-time dependent representation of ice nucleation, which does not account for the stochasticity of the nucleation process. To calculate nₙ both pollens were assumed to have a density of 1 g/cm³. Hornbeam pollen grains were taken to have a diameter of 33 μm² and birch pollen grains 24 μm.¹ These results and literature data from Dreischmeier et al.¹ are presented in FIG. 2 (c). In our experiments nucleation occurs in birch PWW around a degree colder than results generated using essentially identical preparation procedures and measurement techniques in previous work.^{1,3} As shown in FIG. 2 (b), this suggests that the birch PWW used in this study here contains around 10 times fewer nucleators per pollen grain at −16° C. compared to that tested by Dreischmeier et al.¹ while the hornbeam PWW contains similar numbers of nucleators per pollen grain to the Dreischmeier birch PWW. It is known that PWW from the same species can nucleate ice differently⁴ so this is not unexpected.

Our results for freezing of microlitre droplets of PWW do not directly suggest that PWW is capable of nucleating ice at temperatures above −10° C. We then measured the freezing temperature of 100 μL volumes of PWW in SBS 96-well plates, which are relevant for cell cryopreservation.

Accurate measurement of freezing temperatures in 96-well plates is challenging, and to date has only been accomplished using complex infrared thermometry.⁵ We have instead used embedded thermocouples to measure plate temperature during cooling. 92 wells of a 96-well plate were filled with 100 μL volumes of MilliQ® water, birch PWW or hornbeam PWW. The remaining four wells, one in the centre of each quadrant of the 96-well plate, had thermocouples embedded in them using pressure sensitive putty. The plates were then placed onto the cold plate of a Cytiva ViaFreeze Uno (VFU) controlled rate freezer. The temperature of the thermocouples was monitored by a thermocouple reader built into the VFU. The VFU was set to cool the plate at a rate of 2° C./min. Freezing was detected by deviation from the set cooling rate as indicated in FIG. 2 (a). The range of freezing temperatures is also indicated by shaded areas on FIG. 2 (b). It should be noted that this technique detects changes in temperature caused by the latent heat released by the ice crystallization which follows the nucleation event in each water volume, rather than the nucleation event itself. However, nucleation temperature is the factor which determines when ice crystallization occurs.

It is likely that the wells containing thermocouples are consistently cooler than the wells containing water as the heat capacity of the putty used to secure the thermocouple is lower than that of water. Once all wells in the vicinity of a measuring thermocouple are frozen, latent heat will stop being produced and the temperature of the thermocouple will start to return to the temperature set by the controlled rate freezer. As such, we have estimated the range of freezing temperatures as being the period during which rate of cooling is decreasing.

In this way, we estimate that in the 96-well plates we used for this study freezing occurs in pure water between −12° C. and −15° C., in birch PWW between −7.7° C. and −9.6° C. and in hornbeam PWW between −6.0° C. and −8.0° C., which is a substantial increase in a cryobiology context. These ranges are shown in FIG. 2 (b) along with literature data for nucleation temperatures for ‘pure’ water frozen in 96-well plates, as determined by an IR camera-based technique.^{5,6} These literature measurements detected nucleation events between about −13° C. and −22° C., with most events between −15° C. and −17° C., i.e. colder than our measurements. It is likely that the thermocouple technique we have used has limited sensitivity to isolated freezing events, compared to the IR camera technique, which individually detects freezing in each well. This means sporadic warmer and colder events may have occurred in our experiments but were not detected. As regards to the observed difference in freezing temperature between our experiments and literature measurements, it is known that freezing temperatures in 96-well plates are highly variable.⁵ Nevertheless, it is clear that both PWWs are capable of inducing freezing at temperatures above −10° C. in 100 μL droplets, warm enough to expect a benefit for cryopreservation procedures.

The ability of PWW to nucleate ice at temperatures as warm as −6° C. is surprising. Each 100 μL droplet of PWW contains the soluble substances from, on average, approximately 30,000 pollen grains, while 1 μL droplets contain the material from only around 3,000 grains, meaning that the active species at −6° C. is much rarer. Classical nucleation theory allows calculation of the critical nucleus size required for a stable ice crystal to form at a given temperature. It has been suggested that biological ice nucleators are likely to roughly match the size of the ice critical nucleus they cause to form.⁷ In this picture, to induce ice nucleation at −7° C., a nucleating molecule of around 1500 kDa is required. By size exclusion chromatography, the birch nucleator active at around −16° C. has been found to be between 335 and 860 kDa,⁸ larger than required for nucleation at −16° C., but insufficient for nucleation at the temperatures observed here in 96-well plates. It may be that larger nucleators are produced by the plants only rarely or that occasional chance dimer or multimer formation is required to produce the more effective nucleators.

### Example 2: Characterisation of Birch and Hornbeam Pollen Washing Water

Some analysis of relevant properties of the PWW which was used was performed to evaluate its suitability for use as a cryoprotectant.

### Sterility

To ensure that PWW used was sterile, we prepared both birch and hornbeam PWWs in MilliQ® water (not cell culture media which contains antibiotics, and would likely impede microbial growth) and then attempted to culture both types of PWW on agar jelly. After 72 hours, there was no sign of microbial growth confirming that the PWWs were sterile.

### Osmolarity

A freezing point osmometer was used to measure the osmolality of both types of PWW made up in MilliQ® water, which was found in both cases to be 30±5 mOsmol/kg.

### Weights

Samples of both birch and hornbeam PWW were freeze-dried and the residue was weighed, establishing that for both types of PWW approximately 0.7% of the total mass of the samples was soluble material derived from the pollen. It was estimated that 36.6% of birch pollen mass and 32.8% of the hornbeam pollen mass was soluble, and that the mass concentration of the solutions was 6.6 mg/mL and 7.0 mg/mL respectively. By assuming that no dissociated ionic compounds are present in PWW, the average molecular mass of the constituents of PWW could be estimated from the osmolarity of the solution and the mass concentration. We calculated that in both cases the average molecular mass was 230±20 Da. As mentioned above, the birch nucleator has been found to be between 335 and 860 kDa 8 and molecules active at temperatures above −10° C. may be larger still. This demonstrates that much of the content of the PWW is not the ice nucleating polysaccharide but other soluble compounds. The nature of these compounds is not known although it seems reasonable to suppose that monosaccharides are present in significant quantities.

### Example 3: Cytotoxicity of PWW on A549 and Jurkat Cells

We assessed the cytotoxicity of PWW by treating A549 monolayers and Jurkat cells (which we later use for cryopreservation experiments) with serial dilutions of PWW for 24 hours, followed by measurement of cell metabolic activity by resazurin assay. The metabolic activity of Jurkat cells treated with 100% (v/v) PWW for 24 hours was reduced to 90.0% relative to untreated controls. For all other concentrations tested, metabolic activity remained above 99.0%. The metabolic activity of A549 cells decreased to 85.4% when treated with 25% (v/v) PWW, which decreased to 68.1 and 60.0% when higher concentration (50 and 100% (v/v)) solutions of PWW were tested A complimentary cytotoxicity assay, the Live/Dead assay (see Materials and Methods), was also used to assess the effect of PWW on A549 cells. The Live/Dead assay monitors cell health in two ways: through measuring intracellular esterase activity by hydrolysis of calcein-AM to calcein; and by determining plasma membrane stability via the uptake of ethidium homodimer-1 by membrane-compromised cells. Following incubation of A549 monolayers with a serial dilution of PWW for 24 hours, no difference in green fluorescent staining (denoting intracellular esterase activity) or red fluorescent staining (indicating membrane-compromised dead cells) was observed between any test condition and untreated controls, suggesting that cell death was not increased at higher PWW concentrations. During the cryopreservation procedure, the cells are only exposed to liquid PWW for around 10 minutes, insufficient for any cytotoxic effect.

These results indicate that PWW is not cytotoxic to cells.

### Example 4: Cryopreservation of Jurkat and A549 Cells

Two different cell types were selected for cryopreservation experiments: adherent monolayers of A549 (immortalized human lung carcinoma) cells and suspended Jurkat (immortalized human T lymphocyte) cells. These were chosen as they represent two of the most commonly frozen cell formats (monolayers and suspension).

Stable cultures were produced by incubation of cells in appropriate cell media in 96-well plates. FIG. 3 is a schematic diagram of the cryopreservation procedure employed. Briefly, both cell types were exposed to 50 μL cryoprotectant solution containing 10% (v/v) DMSO for A549 cells and 5% (v/v) DMSO for Jurkat cells. This was made up in either complete cell media control or in hornbeam PWW produced from complete cell media as shown in FIG. 1. For each batch of cells, the plates were divided into quadrants: two with the standard DMSO cryoprotectant and two with PWW cryoprotectant. A VFU controlled rate freezer was used to cool the plates from ambient temperatures to −80° C. at nominal controlled rates of 0.5° C./min and 2° C./min. The plates were also placed directly onto the base of a −80° C. chest freezer, a process hereafter referred to as ‘uncontrolled’ freezing, which replicates a method used in a typical lab without access to controlled-rate freezers. After storage for 24 hours at −80° C., the plates were thawed in an incubator held at 37° C. by the addition of 100 μL of warm (37° C.) complete cell media per well. After 10 minutes, the cryopreservation and thawing media was removed and replaced with cell media and the plates were incubated for a further 24 hours at 37° C.

Thermocouples were used in cell freezing experiments to confirm that ice nucleation occurred at a warmer temperature in wells containing PWW compared to those without PWW; and to attempt to directly assess the cooling rate experienced by cells placed directly into the −80° C. freezer. In all cases one thermocouple was embedded in a location surrounded by wells containing PWW and the other thermocouple by wells containing normal cryoprotectant. The observed initial cooling rates were around 0.4° C./min and 1.6° C./min for the 0.5° C./min and 2° C./min nominal cooling rates. For the uncontrolled cooling method, observed initial cooling rate was around 9° C./min, although the cooling rate of the liquid contents of the plate was likely to be somewhat lower. It was clear that nucleation and subsequent freezing occurred at warmer temperatures in the presence of PWW across all cooling rates.

### Post-Thaw Metabolic Activity of Jurkat and A549 Cells

Addition of Hornbeam PWW was found to significantly increase post-thaw metabolic activity, as measured by the resazurin assay, in both A549 (F=809.0, p value=<0.0001) and Jurkat (F=81.51, p value=<0.0001) cells. This result was found to be consistent across all three cooling rates (FIG. 4). Table 3 reports the number of wells frozen in each replicate along with post-thaw metabolic activities.

TABLE 3
Conditions and post-thaw metabolic activity for cryopreservation
experiments, with and without hornbeam PWW.

| | | | | | Post-thaw |
|---|---|---|---|---|---|
| | | | | | metabolic |
| | | | Number | Number | activity with |
| | | | of wells | of wells | PWW (% of |
| | Cooling | | with | without | unfrozen |
| Cell type | rate | Repeat | PWW^{(a)} | PWW | control) ^{(b)} |
| Jurkat | Uncontrolled | 1 | 28 | 30 | 127.8 |
| Jurkat | Uncontrolled | 2 | 30 | 30 | 113.7 |
| Jurkat | Uncontrolled | 3 | 30 | 28 | 94.7 |
| Jurkat | 0.5° C./min | 1 | 30 | 30 | 42.7 |
| Jurkat | 0.5° C./min | 2 | 30 | 30 | 65.1 |
| Jurkat | 0.5° C./min | 3 | 30 | 30 | 90 |
| Jurkat | 2° C./min | 1 | 30 | 30 | 87.8 |
| Jurkat | 2° C./min | 2 | 30 | 30 | 89.4 |
| Jurkat | 2° C./min | 3 | 30 | 30 | 114.8 |
| A549 | Uncontrolled | 1 | 16 | 14 | 87.7 |
| A549 | Uncontrolled | 2 | 30 | 30 | 54.8 |
| A549 | Uncontrolled | 3 | 28 | 28 | 59.1 |
| A549 | 0.5° C./min | 1 | 28 | 28 | 33.0 |
| A549 | 0.5° C./min | 2 | 30 | 29 | 67.7 |
| A549 | 0.5° C./min | 3 | 30 | 30 | 63.1 |
| A549 | 2° C./min | 1 | 22 | 22 | 23.6 |
| A549 | 2° C./min | 2 | 30 | 30 | 75.2 |
| A549 | 2° C./min | 3 | 30 | 30 | 14.1 |

^{(a)}Wells in a 8 × 12 96-well plate,
^{(b)} Metabolic activity measured by resazurin assay for 4 hours at 37° C. and normalised relative to unfrozen controls

The resazurin assay was chosen to allow a sufficiently large dataset to be produced in a reasonable amount of time; however, qualitatively similar results were observed in preliminary studies using trypan blue staining. Some variation was observed between experimental repeats (FIG. 4(a-f)); therefore a linear mixed effect modelling framework was employed to appropriately account for this variation as a random effect. Model selection via Akaike Information Criterion (AIC) revealed that the most parsimonious model for both cell types accounted for both the presence and absence of PWW and the three freezing rates (Tables 4 and 5), demonstrating that both factors had an effect on metabolic activity.

TABLE 4
Model selection table via AIC for A549 cell line.

| Model formula | AIC | ΔAIC^{(a)} | R² value^{(b)} |
|---|---|---|---|
| nMR ~ (1\|exp. no) | 4746.2 | — | 0.029 |
| nMR ~ Pollen + (1\|exp. no) | 4251.9 | −494.3 | 0.653 |
| nMR ~ Freezing rate + (1\|exp. no) | 4737.2 | −9 | 0.052 |
| nMR ~ Pollen + Freezing rate + (1\|exp. | 4220.9 | −525.3 | 0.675 |
| no) | | | |
| nMR ~ Pollen * Freezing rate + | 4187.1 | −559.1 | 0.700 |
| (1\|exp. no) | | | |

^{(a)}ΔAIC values are reported as differences from the most simplistic model. The most parsimonious model is shown in bold. R² squares are reported as the conditional (including both fixed and random effects),
^{(b)}R squared and are calculated using the r.squared.GLMM function in the ‘MuMln’ package⁴⁶ in R.

TABLE 5
Model selection table via AIC for Jurkat cell line.

| Model formula | AIC | ΔAIC^{(a)} | R² value^{(b)} |
|---|---|---|---|
| nMR ~ (1\|exp. no) | 5328.3 | — | 0.005 |
| nMR ~ Pollen + (1\|exp. no) | 5254.0 | −74.3 | 0.137 |
| nMR ~ Freezing rate + (1\|exp. no) | 4946.3 | −382.0 | 0.515 |
| nMR ~ Pollen + Freezing rate + (1\|exp. | 4777.8 | −550.5 | 0.646 |
| no) | | | |
| nMR ~ Pollen * Freezing rate + | 4715.9 | −612.4 | 0.686 |
| (1\|exp. no) | | | |

^{(a)}ΔAIC values are reported as differences from the most simplistic model. The most parsimonious model is shown in bold. R² squares are reported as the conditional (including both fixed and random effects),
^{(b)}R squared and are calculated using the r.squared.GLMM function in the ‘MuMln’ package⁴⁶ in R.

Specifically, metabolic activity was found to be highest in the presence of PWW using the uncontrolled cooling rate for both cell types (Table 6). For A549 cells, use of PWW facilitates successful cryopreservation as post-thaw metabolic activity is close to zero when PWW is absent (FIG. 4(d-f)). For Jurkat cells, PWW increased post-thaw metabolic activity relative to no PWW in all three cooling rates, although the effect was less pronounced at the highest cooling rate. These results clearly demonstrate that the addition of Hornbeam PWW provides a significant increase in post-thaw viability for both cell types following cryopreservation in a convenient 96-well plate format.

TABLE 6
Fixed effect estimates and confidence intervals (lower = 2.5% −
higher = 97.5%) for post-thaw A549 and Jurkat cell metabolic
rate, extracted from the most parsimonious model. All values are
rounded to 2 decimal places.

| | | Cooling rate (° C./min) | | |
|---|---|---|---|---|
| | | 0.5 | 2.0 | Uncontrolled |
| A549 | No PWW | 1.58 | 1.34 | 1.16 |
| | | (−6.63-9.79) | (−6.92-9.58) | (−7.20-9.46) |
| | PWW | 55.04 | 38.64 | 62.83 |
| | | (41.64-68.44) | (25.00-52.25) | (48.76-76.83) |
| Jurkat | No PWW | 31.86 | 63.88 | 104.65 |
| | | (26.50-37.21) | (58.53-69.24) | (99.29-110.01) |
| | PWW | 65.95 | 97.35 | 109.83 |
| | | (55.12-76.78) | (86.52-108.18) | (99.00-120.66) |

To summarize, use of PWW during cryopreservation of suspended Jurkat and adherent A549 cells in 96-well plates gives significant improvements to cell metabolic activity post-thaw, which cannot be achieved using standard cryoprotectants which have no impact on the ice nucleation temperature. For adherent A549 cells, use of PWW effectively enables cryopreservation in 96-well plates as post-thaw metabolism is near-zero in the absence of PWW, while for suspended Jurkat cells it allows near-quantitative cryopreservation, which is a key challenge, especially for related immune cells which form the basis of emerging cell-based therapies such as Chimeric antigen receptor T (CAR-T) cells. We have shown that PWW is capable of nucleating ice at temperatures above −10° C. in 96-well plates and attribute the improved post-thaw outcomes to the increase in nucleation temperatures. Given that PWW is also sterile, as we have demonstrated, it represents a new class of soluble cryoprotectant that can be straightforwardly added to existing cryoprotectant mixtures with reasonable expectation of improved outcomes, particularly when volumes of less than 1 mL are frozen.

### REFERENCES

1 Tomás, R. M. F., Bailey, T. L., Hasan, M. & Gibson, M. I. Extracellular Antifreeze Protein Significantly Enhances the Cryopreservation of Cell Monolayers. Biomacromolecules 20, 3864-3872, doi:10.1021/acs.biomac.9b00951 (2019).
2 Murray, K. A. & Gibson, M. I. Post-Thaw Culture and Measurement of Total Cell Recovery Is Crucial in the Evaluation of New Macromolecular Cryoprotectants. Biomacromolecules 21, 2864-2873, doi:10.1021/acs.biomac.0c00591 (2020).
3 Daily, M. I. et al. Cryopreservation of primary cultures of mammalian somatic cells in 96-well plates benefits from control of ice nucleation. Cryobiology 93, 62-69, doi:https://doi.org/10.1016/j.cryobiol.2020.02.008 (2020).
4 Mazur, P., Leibo, S. P. & Chu, E. H. Y. A two-factor hypothesis of freezing injury. Exp. Cell Res. 71, 345-355, doi:http://dx.doi.org/10.1016/0014-4827(72)90303-5 (1972).
5 Fahy, G. M., Macfarlane, D. R., Angell, C. A. & Meryman, H. T. Vitrification as an Approach to Cryopreservation. Cryobiology 21, 407-426 (1984).
6 Mazur, P. KINETICS OF WATER LOSS FROM CELLS AT SUBZERO TEMPERATURES AND THE LIKELIHOOD OF INTRACELLULAR FREEZING. J Gen Physiol 47, 347-369, doi:10.1085/jgp.47.2.347 (1963).
7 Mazur, P. The role of intracellular freezing in the death of cells cooled at supraoptimal rates. Cryobiology 14, 251-272 (1977).
8 Meneghel, J., Kilbride, P. & Morris, G. J. Cryopreservation as a Key Element in the Successful Delivery of Cell-Based Therapies-A Review. Front Med (Lausanne) 7, 592242-592242, doi:10.3389/fmed.2020.592242 (2020).
9 Mazur, P., Farrant, J., Leibo, S. P. & Chu, E. H. Y. Survival of hamster tissue culture cells after freezing and thawing: Interactions between protective solutes and cooling and warming rates. Cryobiology 6, 1-9, doi:https://doi.org/10.1016/S0011-2240(69)80002-7 (1969).
10 Morris, G. J. & Acton, E. Controlled ice nucleation in cryopreservation—A review. Cryobiology 66, 85-92, doi:10.1016/j.cryobiol.2012.11.007 (2013).
11 Bigg, E. K. The supercooling of water. Proceedings of the Physical Society. Section B 66, 688 (1953).
12 Lauterboeck, L., Hofmann, N., Mueller, T. & Glasmacher, B. Active control of the nucleation temperature enhances freezing survival of multipotent mesenchymal stromal cells. Cryobiology 71, 384-390, doi:https://doi.org/10.1016/j.cryobiol.2015.10.145 (2015).
13 Petersen, A., Schneider, H., Rau, G. & Glasmacher, B. A new approach for freezing of aqueous solutions under active control of the nucleation temperature. Cryobiology 53, 248-257, doi:https://doi.org/10.1016/j.cryobiol.2006.06.005 (2006).
14 Lauterboeck, L., Wieprecht, W., Kabelac, S. & Glasmacher, B. Laser contactless ice nucleation for stem cell cryopreservation. Cryobiology 73, 419, doi:https://doi.org/10.1016/j.cryobiol.2016.09.081 (2016).
15 Weng, L., Tessier, S. N., Swei, A., Stott, S. L. & Toner, M. Controlled ice nucleation using freeze-dried Pseudomonas syringae encapsulated in alginate beads. Cryobiology 75, 1-6, doi:https://doi.org/10.1016/j.cryobiol.2017.03.006 (2017).
16 Teixeira, M. et al. Ice nucleating agents allow embryo freezing without manual seeding. Theriogenology 104, 173-178, doi:https://doi.org/10.1016/j.theriogenology.2017.08.012 (2017).
17 Massie, I., Selden, C., Hodgson, H. & Fuller, B. Cryopreservation of encapsulated liver spheroids for a bioartificial liver: reducing latent cryoinjury using an ice nucleating agent. Tissue Eng Part C Methods 17, 765-774, doi:10.1089/ten.TEC.2010.0394 (2011).
18 Kojima, T., Soma, T. & Oguri, N. Effect of ice nucleation by droplet of immobilized silver iodide on freezing of rabbit and bovine embryos. Theriogenology 30, 1199-1207, doi:https://doi.org/10.1016/0093-691X(88)90296-8 (1988).
19 Wragg, N. M., Tampakis, D. & Stolzing, A. Cryopreservation of Mesenchymal Stem Cells Using Medical Grade Ice Nucleation Inducer. International Journal of Molecular Sciences 21, 8579 (2020).
20 Jiang, B. et al. Sand-mediated ice seeding enables serum-free low-cryoprotectant cryopreservation of human induced pluripotent stem cells. Bioactive Materials 6, 4377-4388, doi:https://doi.org/10.1016/j.bioactmat.2021.04.025 (2021).
21 Diehl, K., Matthias-Maser, S., Jaenicke, R. & Mitra, S. K. The ice nucleating ability of pollen: Part II. Laboratory studies in immersion and contact freezing modes. Atmos. Res. 61, 125-133 (2002).
22 von Blohn, N., Mitra, S. K., Diehl, K. & Borrmann, S. The ice nucleating ability of pollen: Part III: New laboratory studies in immersion and contact freezing modes including more pollen types. Atmos. Res. 78, 182-189 (2005).
23 Pummer, B. G., Bauer, H., Bernardi, J., Bleicher, S. & Grothe, H. Suspendable macromolecules are responsible for ice nucleation activity of birch and conifer pollen. Atmos. Chem. Phys. 12, 2541-2550, doi:10.5194/acp-12-2541-2012 (2012).
24 Pummer, B. G. et al. Ice nucleation by water-soluble macromolecules. Atmos. Chem. Phys. 15, 4077-4091, doi:10.5194/acp-15-4077-2015 (2015).
25 Gute, E. & Abbatt, J. P. D. Ice nucleating behavior of different tree pollen in the immersion mode. Atmos. Environ. 231, 117488, doi:https://doi.org/10.1016/j.atmosenv.2020.117488 (2020).
26 Augustin, S. et al. Immersion freezing of birch pollen washing water. Atmos. Chem. Phys. 13, 10989-11003, doi:10.5194/acp-13-10989-2013 (2013).
27 Gute, E. & Abbatt, J. P. D. Oxidative Processing Lowers the Ice Nucleation Activity of Birch and Alder Pollen. Geophys. Res. Lett. 45, 1647-1653, doi:https://doi.org/10.1002/2017GL076357 (2018).
28 Gute, E., David, R. O., Kanji, Z. A. & Abbatt, J. P. D. Ice Nucleation Ability of Tree Pollen Altered by Atmospheric Processing. ACS Earth and Space Chemistry 4, 2312-2319, doi:10.1021/acsearthspacechem.0c00218 (2020).
29 O'Sullivan, D. et al. The relevance of nanoscale biological fragments for ice nucleation in clouds. Sci. Rep. 5, doi:10.1038/srep08082 (2015).
30 Dreischmeier, K., Budke, C., Wiehemeier, L., Kottke, T. & Koop, T. Boreal pollen contain ice-nucleating as well as ice-binding ‘antifreeze’ polysaccharides. Scientific Reports 7, 41890, doi:10.1038/srep41890 (2017).
31 Marcolli, C., Nagare, B., Welti, A. & Lohmann, U. Ice nucleation efficiency of AgI: review and new insights. Atmos. Chem. Phys. 16, 8915-8937, doi:10.5194/acp-16-8915-2016 (2016).
32 Vonnegut, B. THE NUCLEATION OF ICE FORMATION BY SILVER IODIDE. J. Appl. Phys. 18, 593-595, doi:10.1063/1.1697813 (1947).
33 Harrison, A. D. et al. Not all feldspars are equal: a survey of ice nucleating properties across the feldspar group of minerals. Atmos. Chem. Phys. 16, 10927-10940, doi:10.5194/acp-16-10927-2016 (2016).
34 Atkinson, J. D. et al. The importance of feldspar for ice nucleation by mineral dust in mixed-phase clouds. Nature 498, 355-358, doi:10.1038/nature12278 (2013).
35 Whale, T. F. et al. A technique for quantifying heterogeneous ice nucleation in microlitre supercooled water droplets. Atmos. Meas. Tech. 8, 2437-2447, doi:10.5194/amt-8-2437-2015 (2015).
36 Halbritter, H., Schneider, H., Sam, S. & Auer, W. Carpinus betulus. In: PalDat—A palynological database., <https://www.paldat.org/pub/Carpinus_betulus/306095;jsessionid=BEE062A79220482A D5DD0BD482E3B404> (2021).
37 Harrison, A. D. et al. An instrument for quantifying heterogeneous ice nucleation in multiwell plates using infrared emissions to detect freezing. Atmos. Meas. Tech. 11, 5629-5641, doi:10.5194/amt-11-5629-2018 (2018).
38 Wex, H. et al. Intercomparing different devices for the investigation of ice nucleating particles using Snomax® as test substance. Atmos. Chem. Phys. 15, 1463-1485, doi:10.5194/acp-15-1463-2015 (2015).
39 Turner, M. A., Arellano, F. & Kozloff, L. M. Three separate classes of bacterial ice nucleation structures. J. Bacteriol. 172, 2521-2526, doi:10.1128/jb.172.5.2521-2526.1990 (1990).
40 Baboo, J. et al. The Impact of Varying Cooling and Thawing Rates on the Quality of Cryopreserved Human Peripheral Blood T Cells. Scientific Reports 9, 3417, doi:10.1038/s41598-019-39957-x (2019).
41 Corsini, J., Maxwell, F. & Maxwell, I. H. Storage of various cell lines at −70 C or −80 C in multi-well plates while attached to the substratum. BioTechniques 33, 42-46 (2002).
42 Ebertz, S. L. & McGann, L. E. Cryoinjury in endothelial cell monolayers. Cryobiology 49, 37-44, doi:https://doi.org/10.1016/j.cryobiol.2004.04.003 (2004).
43 Rutt, T. et al. Thermal expansion of substrate may affect adhesion of Chinese hamster fibroblasts to surfaces during freezing. Cryobiology 86, 134-139, doi:https://doi.org/10.1016/j.cryobiol.2018.10.006 (2019).
44 Eskandari, N., Marquez-Curtis, L. A., McGann, L. E. & Elliott, J. A. W. Cryopreservation of human umbilical vein and porcine corneal endothelial cell monolayers. Cryobiology 85, 63-72, doi:10.1016/j.cryobiol.2018.10.001 (2018).
45 Bates, D., Mächler, M., Bolker, B. & Walker, S. Fitting Linear Mixed-Effects Models Using Ime4. 2015 67, 48, doi:10.18637/jss.v067.i01 (2015).
46 Bartoń, K. MuMln: Multi-Model Inference. R package version 1.43.17., <https://CRAN.R-project.org/package=MuMln> (2020).

1. A process for producing a frozen cryopreserving composition comprising biological material, the process comprising the step:
(a) freezing biological material in a cryopreserving composition at a cryopreserving temperature,
wherein the cryopreserving composition comprises:
(i) an aqueous solution; and
(ii) ice-nucleating agents obtained or obtainable from pollen or spores.

2. A method for improving cell recovery from biological material which has been cryopreserved, wherein the biological material comprises cells, the method comprising the steps:
(a) storing the biological material in a cryopreserving composition at a cryopreserving temperature; and optionally
(b) thawing the biological material;
wherein the cryopreserving composition comprises:
(i) an aqueous solution; and
(ii) ice-nucleating agents obtained or obtainable from pollen or spores.

3. The process as claimed in claim 1 wherein the cryopreserving composition additionally comprises:
(iii) a cryoprotectant, or DMSO.

4. The process as claimed in claim 1, wherein the biological material comprises:
(a) cells, tissues, whole organs or parts of organs, or
(b) polypeptides or nucleic acids.

5. The process as claimed in claim 1, wherein:
(a) the presence of the ice-nucleating agents in the cryopreserving composition raises the ice nucleating temperature at least 2° C., or at least 3, 4, or 5° C. (compared to a control cryopreserving composition without the ice-nucleating agents); or
(b) the presence of the ice-nucleating agents in the cryopreserving composition raises the ice nucleating temperature to at least −10° C., or to at least −9° C., −8° C., −7° C. or −6° C.

6. The process as claimed in claim 1, wherein the ice-nucleating agents are obtained or are obtainable from pollen from a plant species selected from the group consisting of Sambucus nigra, Hymenocallis littoralis, Araucaria araucana, Hyacinthus orientalis, Betula pendula, Carpinus betulus, Corylus avellana, Alnus glutinosa, Cistus populifoloius, Sequoiadendron giganteum, Cupressus sempervirens, Erica multiflora, Crocus vernus, Clerodendrum speciosissimum, Fritillaria meleagris, Musa rubra, Nymphaea ‘Kew's Stowaway Blues’, Pinus halepensis, Pinus coulteri, Pinus mugo, Picea brachytyla, Picea wilsonii, Plantago lanceolata, Cynosurus cristatus, Dactylis glomerata, Arundo formosana, Aesculus hippocastanum, Encephalartos equatoralis, or Hedychium coronarium.

7. The process as claimed in claim 1, wherein the ice-nucleating agents are provided in the form of an infusion, or an aqueous infusion, which has been obtained from the pollen or spores, or from pollen washing water (PWW).

8. The process as claimed in claim 1, wherein the ice-nucleating agents are ones which are capable of passing through a filter with a maximum pore size of 150 μm, 200 nm or 3 nm.

9. The process as claimed in claim 1, wherein the cryopreserving composition comprises ice nucleating agents which have been obtained from about 10,000 Betula pendula or Carpinus betulus pollen grains, or from about 1,000-2,000 Pinus mugo pollen grains.

10. The process as claimed in claim 1, wherein the ice nucleating agents are polysaccharides, or polysaccharides having molecular weights of 50-400 kDa, or 100-300 kDa.

11. The process as claimed in claim 1, wherein the freezing or storing of the biological material in a cryopreserving composition is performed in one or more wells of a microtitre plate.

12. The process as claimed in claim 1 wherein the volume of the cryopreserving composition to be frozen is 1 μl to 1 ml, or 1 ml to 1 litre, or 25-400 μl.

13. (canceled)

14. A cryopreserving composition comprising:
(i) ice-nucleating agents obtained or obtainable from pollen or spores; and
(ii) a cryoprotectant, or DMSO.

15. The cryopreserving composition as claimed in claim 14, additionally comprising:
(iii) biological material,
optionally wherein the cryopreserving composition is at a cryopreserving temperature.

16. A kit comprising:
(i) ice-nucleating agents obtained or obtainable from pollen or spores; and
(ii) a cryoprotectant; and optionally
(iii) instructions for use of (i) and (ii) in a process as claimed in claim 1.

17. (canceled)
